(19) United States
(12) Patent Application Publication
(10) Pub. No.: US 2026/0026965 A1
(43) Pub. Date: Jan. 29, 2026

(54) Gel Pad Assembly Using Free Rotatable Fluid Joints

(71) Applicant: C. R. Bard, Inc., Franklin Lakes, NJ (US)

(72) Inventors: Qihua Xu, Cary, NC (US); Eric A. Fallows, Apex, NC (US); Adam T. Martin, Holly Springs, NC (US); Nicholas J. Jardine, Holly Springs, NC (US); Sean E. Walker, Platteville, CO (US)

(21) Appl. No.: 19/351,132

(22) Filed: Oct. 6, 2025

Related U.S. Application Data
(63) Continuation of application No. 17/678,965, filed on Feb. 23, 2022, now Pat. No. 12,433,785.
(60) Provisional application No. 63/152,733, filed on Feb. 23, 2021.

(51) Int. Cl. A61F 7/08; A61F 7/00; A61F 7/02

(52) U.S. Cl. CPC A61F 7/08; A61F 7/02; A61F 2007/0054; A61F 2007/0219; A61F 2007/0244

(57) ABSTRACT

A medical pad for exchanging thermal energy between a targeted temperature management (TTM) fluid and a patient is disclosed. The pad can include a fluid containing layer and a fluid conduit attached therewith via rotatable joint where the joint is configured to facilitate rotation of the fluid conduit with respect to the pad. A method of manufacturing the pad can include coupling a first member of the rotatable joint to a fluid delivery line, coupling the second member to the fluid containing layer, and inserting the first member within an opening of the second member to facilitate a snap-fit retention mechanism between first member and the second member. A method of using the pad can include rotating the fluid delivery line relative to the thermal pad via the rotatable joint when initially applying the pad to the patient or when adjusting an orientation of the pad on the patient.

### PRIORITY

This application is a continuation of U.S. patent application Ser. No. 17/678,965, filed Feb. 23, 2022, now U.S. Pat. No. 12,433,785, which claims the benefit of priority to U.S. Provisional Application No. 63/152,733, filed Feb. 23, 2021, each of which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled with a patient to affect surface-to-surface thermal energy exchange with the patient. In general, TTM systems comprise a TTM fluid control module coupled with at least one contact pad via a fluid deliver line. One such TTM system is disclosed in U.S. Pat. No. 6,645,232, titled, “Patient Temperature Control System with Fluid Pressure Maintenance,” and one such thermal contact pad and related system is disclosed in U.S. Pat. No. 6,197,045 (the '045 patent), titled, “Cooling/heating Pad and System,” both of which are incorporated herein by reference in their entireties. As noted in the '045 patent, the ability to establish and maintain thermally intimate pad-to-patient contact is of importance to fully realizing medical efficacies with TTM systems. In some instances, the location and/or orientation of the pad may need to be adjusted to maximize thermal energy exchange with the patient.

The pad is generally coupled with the TTM control module via the fluid delivery line where the fluid delivery to be attached to the pad in a fixed orientation. In some instances, the fixed orientation may make it difficult for the clinician to apply the pad to the patient in the desired position and/or orientation with the fluid delivery line extending away from the pad in a fixed direction. As such, it would be advantageous for the clinician to rotate the fluid delivery line with respect to the pad when initially applying the pad to the patient and/or when adjusting the position of the pad after initial application. Disclosed herein are embodiments of devices and methods for adjusting an orientation of the fluid delivery line with respect to the thermal contact pad when performing the TTM therapy.

### SUMMARY OF THE INVENTION

Briefly summarized, disclosed herein is a medical pad for exchanging thermal energy between a targeted temperature management (TTM) fluid and a patient. The pad includes a fluid containing layer having a top side and a bottom side, where the fluid containing layer is configured for containing the TTM fluid. The fluid containing also includes a fluid inlet and a fluid outlet, and the TTM fluid is circulatable within the fluid containing layer from the fluid inlet to the fluid outlet. A fluid conduit is attached to one of the fluid inlet or the fluid outlet via a rotatable joint, where the joint is configured to facilitate rotation of the fluid conduit with respect to the pad. The joint includes a lumen extending through the joint, where the lumen establishes fluid communication between the fluid conduit and the fluid containing layer.

In some embodiments, the joint includes a first joint member attached to the fluid conduit and a second joint member attached to the fluid containing layer, and the first joint member is rotatable with respect to the second joint member. The first joint member may include an elbow configured to establish parallel orientation of a distal portion of the fluid conduit with respect to the pad. The second joint member may be attached to an outside surface of the fluid containing layer.

In some embodiments, a portion of one of the first joint member or the second joint member is disposed with an opening of the other one of the first joint member or the second joint member, and in further embodiments, the joint includes a sealing member disposed between the first joint member and the second joint member. The sealing member is configured to define a dynamic fluid seal between the first joint member and the second joint member. One of the first joint member or the second joint member may include an annular groove containing the sealing member, and the other one of the first joint member or the second joint member may include annular sealing surface disposed in interfering contact with the sealing member. During rotation of the first joint member with respect to the second joint member, the annular sealing surface may be displaced with respect to the sealing member, and the joint may include a lubricant disposed between the annular sealing surface and the sealing member. During assembly of the first joint member with the second member, the annular sealing surface may be longitudinally displaced with respect to the sealing member.

The sealing member may be formed of a toroidal shape and in some embodiments, the sealing member is an O-ring (e.g., a packing or toric joint, which may comprise a mechanical gasket formed of, for example, elastomer). In some embodiments, the joint includes more than one sealing member.

The first joint member is configured to couple with the second joint member via a snap-fit retention mechanism and the retention mechanism may include at least one deflectable member. The retention mechanism may be disposed within the opening, and the second joint member may include the opening.

In some embodiments, the opening includes a first inside annular surface having a first diameter, the first joint member includes a first outside annular surface, and the first outside annular surface is disposed in sliding engagement with the first inside annular surface. In further embodiments, the opening includes a second inside annular surface having a second diameter, the first joint member includes a second outside annular surface, and the second outside annular surface is disposed in sliding engagement with the second inside annular surface. The first inside annular surface may be disposed adjacent a top side of the second joint member, and the second diameter may be less than the first diameter. The opening may also include a recess extending upward from a bottom side of the second joint member, where the recess defines a third diameter, and the third diameter may be greater than the second diameter.

The first joint member may include the deflectable member of the retention mechanism and the deflectable member may be biased radially outward from the lumen. When the first joint member is coupled with the second joint member, at least a portion of the deflectable member may be disposed within the recess.

The joint may be configured to facilitate assembly of the first joint member with the second member via longitudinal displacement of the first joint member with respect to the second joint member. The deflectable member may include an angled surface configured to engage the second annular surface during assembly of the first joint member with the second joint member, and during assembly, engagement of the angled surface may cause the deflectable member to deflect radially inward toward the lumen. In some embodiments, the retention mechanism includes more than one deflectable member.

The pad may include an insulation layer disposed on the top side of the fluid containing layer, and the joint may be disposed through an opening in the insulation layer. In some embodiments, at least a portion of the second joint member is disposed between the insulation layer and the fluid containing layer.

Also disclosed herein is a method of manufacturing a thermal pad for providing thermal energy exchange with a patient. The method includes providing a fluid containing layer having top side and a bottom side. The fluid containing layer is configured for containing the TTM fluid. The fluid containing layer also includes a fluid inlet and a fluid outlet, and the TTM fluid is circulatable within the fluid containing layer from the fluid inlet to the fluid outlet. Also provided is a first member of a rotatable joint and a second member of the rotatable joint.

The method further includes coupling the first member to a fluid delivery line, coupling the second member to the fluid containing layer, and inserting the first member within an opening of the second member, where inserting the first member within an opening of the second member facilitates a snap-fit retention mechanism between the first member and the second member, and the retention mechanism prevents separation of the second member from the first member.

The method of manufacturing may further include placing a sealing member within a groove of one of the first member or the second member.

In some embodiments of the method of manufacturing, one of the first member or the second member includes a deflectable member of the retention mechanism, where the deflectable member includes an angled surface, and the other one of the first member or the second member includes a contact surface so that during insertion, the angled surface engages the contact surface to urge the deflectable member away from the contact surface.

In some embodiments, at least one of the steps of coupling the first member to a fluid delivery line or coupling the second member to the fluid containing layer is performed at a first facility, and the step of inserting the first member within an opening of the second member is performed at a second facility. The first facility may be a manufacturing facility and the second facility may be a patient care facility.

Also disclosed herein is a method of using a thermal pad assembly configured for providing thermal energy exchange with a patient. The method includes providing a thermal pad assembly including a thermal pad and a fluid delivery line coupled with the thermal pad. The method further includes rotating the fluid delivery line relative to the thermal pad via a rotatable joint disposed between the fluid delivery line and the thermal pad to establish a first orientation of the fluid delivery line relative to the thermal pad. The method further includes applying the thermal pad to the patient at a first angular orientation of the thermal pad, and coupling the fluid delivery line with a targeted temperature management module.

The method may further include adjusting the angular orientation of the thermal pad away from the first angular orientation toward a second angular orientation and the rotating the fluid delivery line relative to the thermal pad away from the first orientation toward a second orientation of the fluid delivery line relative to the thermal pad.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a targeted temperature management (TTM) system for cooling or warming a patient, in accordance with some embodiments.

FIG. 2 is a top view of the thermal contact pad of FIG. 1, in accordance with some embodiments.

FIG. 3A is a front perspective view of a fluid line member of the rotatable joint of FIGS. 1 and 2, in accordance with some embodiments.

FIG. 3B is detail cross-sectional detail view of a vertical section of the fluid line member of FIG. 3A, in accordance with some embodiments.

FIG. 4A is a front perspective view of a pad member of the rotatable joint of FIGS. 1 and 2, in accordance with some embodiments.

FIG. 4B is a cross-sectional view of the pad member of FIG. 4A, in accordance with some embodiments.

FIG. 5 is a cross-sectional view of a portion of the thermal pad of FIG. 2 cut along sectioning lines 5-5 illustrating the rotatable joint in an assembled state, in accordance with some embodiments.

FIG. 6A is an exploded perspective view of a TTM fluid filter, in accordance with some embodiments.

FIG. 6B is a cross-sectional side view of the filter of FIG. 6A, in accordance with some embodiments.

FIG. 6C is a cross-sectional detail view of the thermal contact pad of FIG. 2 incorporating the filter of FIG. 6A, in accordance with some embodiments.

FIG. 7 is a top view of the thermal contact pad of FIG. 2 disposed in a packaging configuration, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” “horizontal,” “vertical” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled with” refer to any form of interaction between two or more entities, including mechanical, electrical, magnetic, electromagnetic, fluid, signal, communicative (including wireless), and thermal interaction. Two components may be connected to or coupled with each other even though they are not in direct contact with each other. For example, two components may be coupled with each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

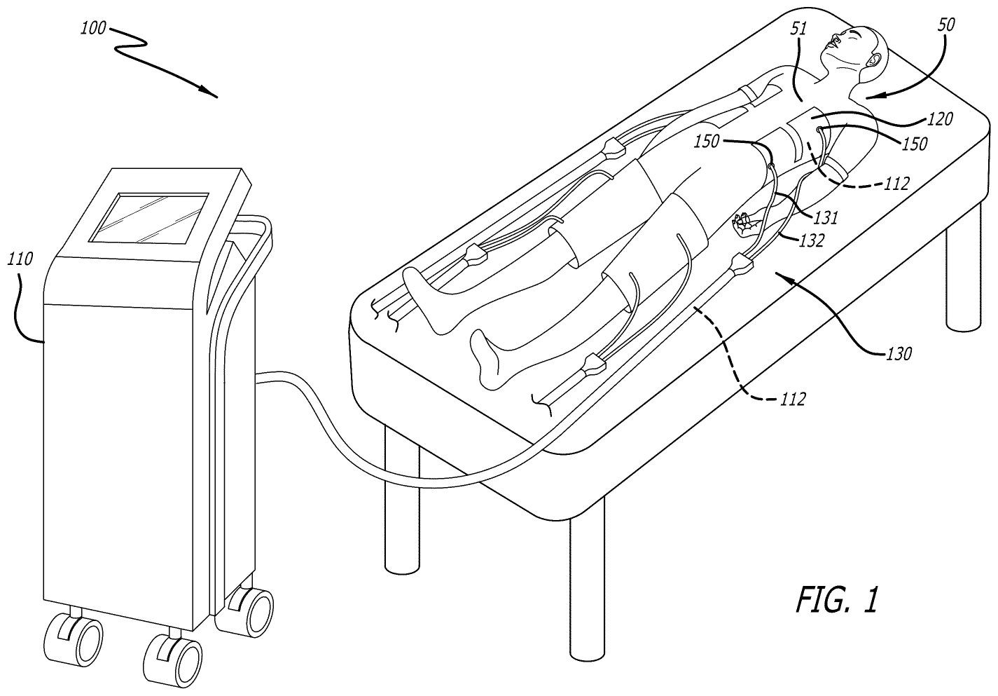

FIG. 1 illustrates a targeted temperature management (TTM) system 100 connected to a patient 50 for administering targeted temperature management therapy to the patient 50 which may include a cooling and/or warming of the patient 50, in accordance with some embodiments. The TTM system 100 includes a TTM module 110 and a thermal contact pad (pad) 120. A fluid delivery line (FDL) 130 coupled with the thermal contact pad 120 extends between the TTM module 110 and the pad 120 to provide of flow of TTM fluid 112 between the TTM module 110 and the pad 120. The FDL includes two fluid conduits to facilitate delivery and return flow of TTM fluid 112 between the TTM module 110 and the pad 120 as further described below.

The TTM system 100 may include 1, 2, 3, 4 or more pads 120 and the TTM system 100 may include 1, 2, 3, 4 or more fluid delivery lines 130. In use, the TTM module 110 prepares the TTM fluid 112 for delivery to the pad 120 by heating or cooling the TTM fluid 112 to a defined temperature in accordance with a prescribed TTM therapy. The TTM module 110 circulates the TTM fluid 112 along a TTM fluid flow path including within the pad 120. The pad 120 is applied to the skin 51 of the patient to facilitate thermal energy exchange between the pad 120 and the patient 50. During the TTM therapy, the TTM module 110 may continually control the temperature of the TTM fluid 112 toward a target TTM temperature.

The FDL 130 includes at least a fluid delivery conduit 131 and a fluid return conduit 132. In use, the TTM fluid 112 may flow from the TTM module 110 through the fluid delivery conduit 131 to the pad 120. The TTM fluid 112 may then flow from thermal pad 120 through the fluid return conduit 132 to the TTM module 110. In some embodiments, the fluid delivery conduit 131 and the fluid return conduit 132 may be attached together along a portion of a length of the FDL 130. The fluid delivery conduit 131 and the fluid return conduit 132 may be separated from each other at each end of the FDL 130.

The FDL 130 is coupled with thermal pad 120 via one or more rotatable joints 150. More specifically each of the fluid delivery conduit 131 and a fluid return conduit 132 is coupled with the thermal pad 120 via a rotatable joint 150.

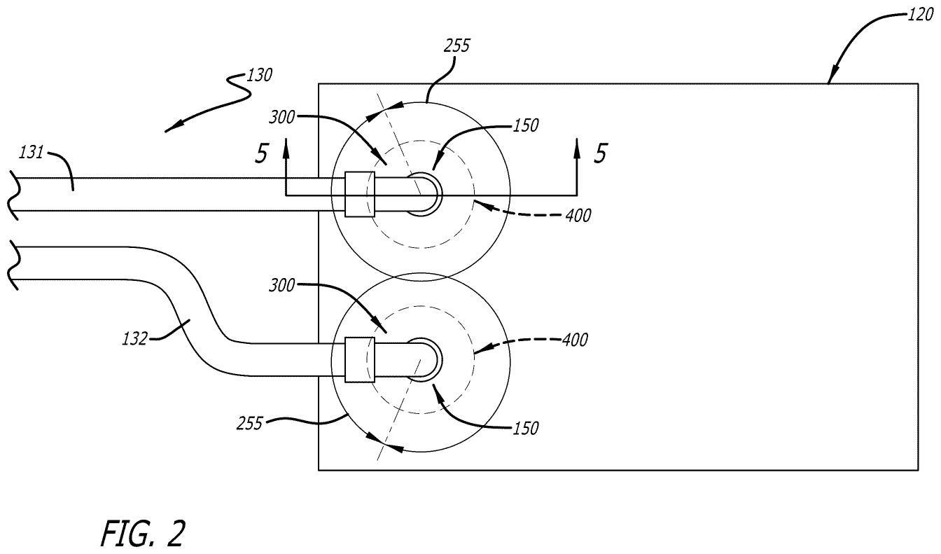

FIG. 2 shows a top view of the pad 120 in accordance with some embodiments. In the illustrated embodiment, the pad 120 includes two rotatable joints 150 coupled between the FDL 130 and the pad 120. As illustrated, the rotatable joints 150 may provide for a rotatable connection between the FDL 130 and the pad 120. The rotatable connection may provide for the FDL 130, or more specifically each of the fluid delivery conduit 131 and the fluid return conduit 132, to rotate. In the illustrated embodiment, each rotatable joint 150 facilitates rotation of each of the fluid delivery conduit 131 and a fluid return conduit 132 through an angle 255 that is unlimited. In other embodiments, the rotatable joint 150 may limit rotation of the FDL 130 to about 90 degrees, 180 degrees, 270, or 360 degrees.

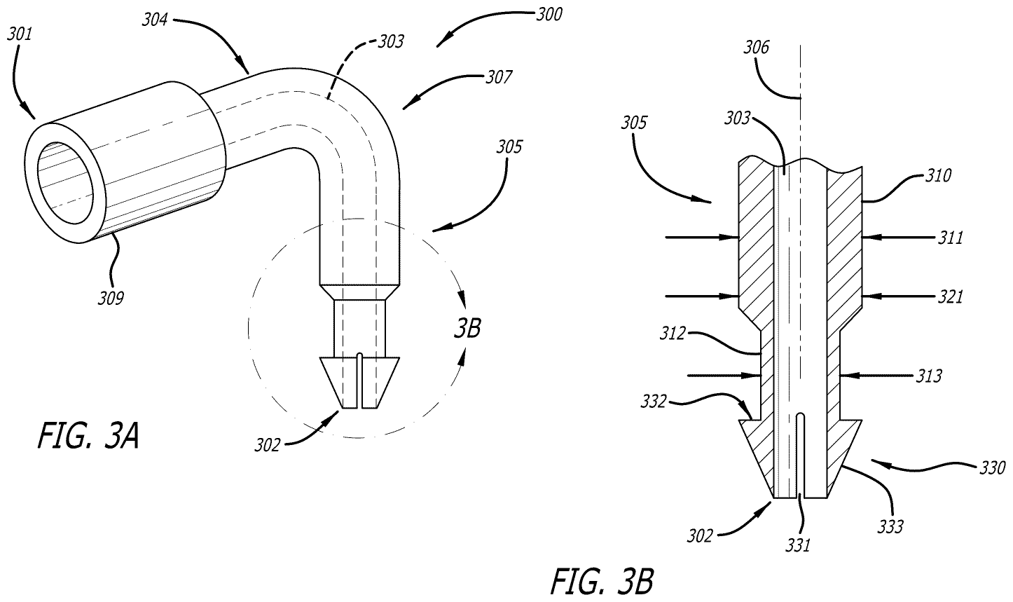

FIGS. 3A-5 illustrate components and functionality of the joint 150. In the illustrated embodiment of FIG. 2, the thermal pad 120 includes two rotatable joints 150 coupled generally to the FDL 130, and more specifically, each of the two rotatable joints 150 are individually coupled with one of the fluid delivery conduit 131 and the fluid return conduit 132. In other embodiments, the pad 120 may include more than two rotatable joints 150 couple to additional fluid conduits. While FIGS. 3-5 and the corresponding description that follows, show and describe the components and functionality of a single joint 150 of FIG. 2, the FIGS. 3-5 and the corresponding description may apply equally well to other and/or all rotatable joints 150.

FIG. 3A is a front perspective view of a conduit member 300 (sometimes referred to as a first joint member) having the general form a fluid fitting with an elbow. The conduit member 300 defines a proximal end 301, a distal end 302, and a fluid lumen 303 extending through the conduit member 300 from the proximal end 301 to the distal end 302. For the purposes of description, the conduit member 300 defines a horizontal section 304 and a vertical section 305. The conduit member 300 includes an elbow 307. A conduit attachment member 309 disposed at the proximal end 301 facilitates fluid coupling of the conduit member 300 with a fluid conduit, (i.e., the fluid delivery conduit 131, the fluid return conduit 132, or any other fluid conduit). In some embodiments, the conduit attachment member 309 is configured to couple with an outside annular surface of the fluid conduit. In other embodiments, the conduit attachment member 309 may couple with a luminal surface of the fluid conduit.

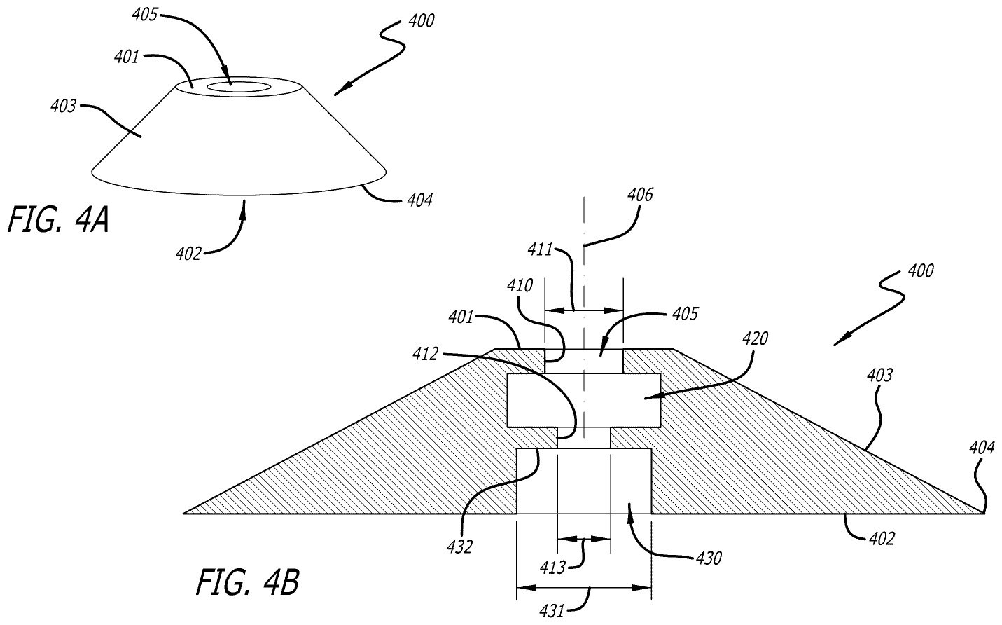

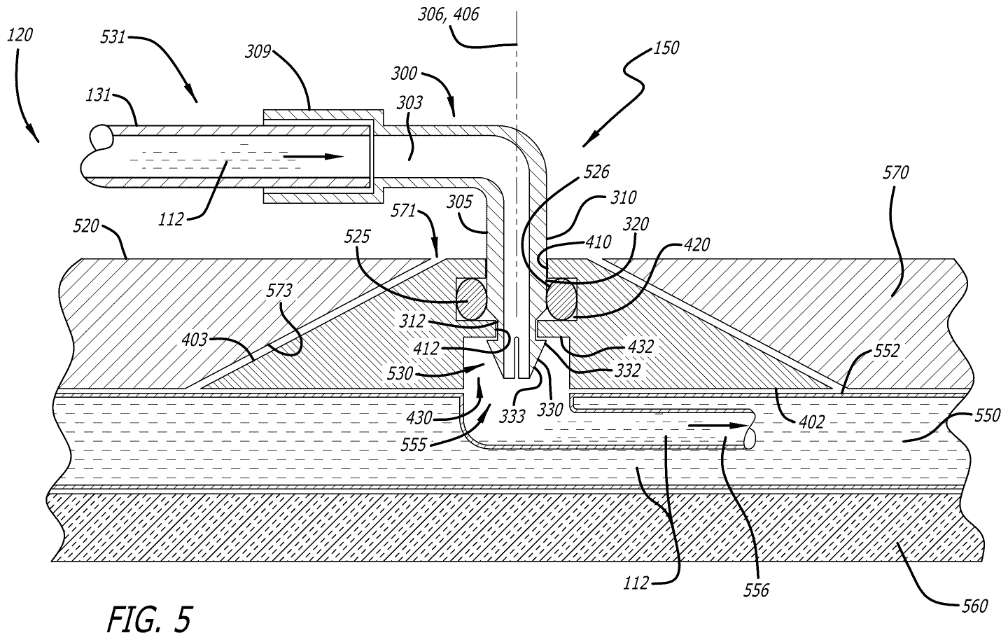

FIG. 3B is a side cross-sectional detail view of a portion of the vertical section 305 of the conduit member 300. The vertical section 305 defines a longitudinal axis 306 which may extend through the center of the vertical section 305. The conduit member 300 includes various features, extending along the vertical section 305, configured to interact with corresponding features of a pad member 400 (see FIGS. 4A, 4B, and 5).

The conduit member 300 includes a first outside bearing surface 310 disposed on an outside circumference of the vertical section 305 defining a first diameter 311. The conduit member 300 also may include a second outside bearing surface 312 disposed on an outside circumference of the vertical section 305 defining a second diameter 312. In some embodiments, the second diameter 312 may be less than the first diameter 311. In other embodiments, the second outside bearing surface 312 may be omitted. In some embodiments, the longitudinal axis 306 may extend through the center of one or both of the first outside bearing surface 310 and the second outside bearing surface 312.

The conduit member 300 further includes an annular sealing surface 320 disposed on an outside circumference of the vertical section 305 defining a third diameter 321. The third diameter 321 may be equal to or less than the first diameter 311. The third diameter 321 may also be equal to or greater than the second diameter 312. In the illustrated embodiment, the annular sealing surface 320 is positioned between the first outside bearing surface 310 and the second outside bearing surface 312. In other embodiments, the annular sealing surface 320 may be positioned proximal the first outside bearing surface 310 or distal the second outside bearing surface 312. In some embodiments, the longitudinal axis 306 may extend through the center of the annular sealing surface 320.

The conduit member 300 includes a deflectable member 330. The deflectable member 330 may be configured to deflect inward toward the central axis 306 or the lumen 303. In other embodiments, the deflectable member 330 may be configured to deflect in an outward or lateral direction. In the illustrated embodiment, the deflectable member 330 is positioned distal the second outside bearing surface 312. In other embodiments, the deflectable member 330 may be positioned at any other location along the vertical section 305 with respect to any of the first outside bearing surface 310, the second outside bearing surface 312, and the sealing surface 320. In some embodiments, the conduit member 300 may include a slit 331 extending through a wall of the vertical section 305 to define a flexible portion of the vertical section 305 and thereby facilitate deflection of the deflectable member 330. In some embodiments, the conduit member 300 may include a single deflectable member 330. In other embodiments, the conduit member 300 may include multiple deflectable members 330. The deflectable member 330 may include a hook 332 and an angled surface 333 for interaction with corresponding features of the pad member 400 as further described below.

FIGS. 4A-4B illustrate a perspective view and a cross-sectional view of a pad member 400 (sometimes referred to as a second joint member) of the rotatable joint 150, and FIG. 5, discussed below, illustrates a cross-sectional view of the pad 120, which provides a detailed illustration of the pad member 400. The pad member 400 may be formed of a flattened conical shape defining a top surface 401 at a proximal end, a bottom surface 402 at a distal end, and a conical surface 403 extending radially outward to a circumference edge 404. The pad member 400 includes various features, disposed within an opening 405, configured to interact with corresponding features of the conduit member 300 (see FIGS. 3A, 3B). The opening 405 defines a longitudinal axis 406 of the pad member 400.

The pad member 400 includes a first inside bearing surface 410 disposed on an inside circumference of the opening 405 defining a first inside diameter 411. The pad member 400 may also include a second inside bearing surface 412 disposed on an inside circumference of the opening 405 defining a second diameter 412. In some embodiments, the second diameter 412 may be less that the first diameter 411 . . . . In some embodiments, the longitudinal axis 406 may extend through the center of one or both of the first inside bearing surface 410 and the second inside bearing surface 412. In other embodiments, the second inside bearing surface 412 may be omitted.

The pad member 400 further includes a groove 420 disposed on an inside surface of the opening 405. In the illustrated embodiment, the groove 420 is positioned between the first inside bearing surface 410 and the second inside bearing surface 412. In other embodiments, the groove 320 may be positioned proximal the first inside bearing surface 410 or distal the second inside bearing surface 412.

The pad member 400 may further include a recess 430 disposed with the opening 405 adjacent the bottom surface 402. The recess 430 may define an inside diameter 431 that is greater than the second inside diameter 413 to define a ledge 432.

The conduit member 300 and the pad member 400 may be formed of the same material or different materials, including polyvinyl chloride (PVC), polypropylene, polyethylene, nylon-6 (polycaprolactam), Nylon 6-6, polyamides (PA), polycarbonates (PC), acrylonitrile butadiene styrene copolymer (ABS), polybutylene terephthalate (PBT), polyethylene terephthalate (PET), polyoxymethylene (POM), or any other suitable medical grade material.

FIG. 5 is a cross-sectional view of the pad 120 cut along sectioning lines 5-5 (see FIG. 2) illustrating a cross-sectional view of the rotatable joint 150. As illustrated, the vertical section 305 of the conduit member 300 is disposed within the opening 405 of the pad member 400. When assembled, the longitudinal axis 306 of the conduit member 300 may be coincident with the longitudinal axis 406 of the pad member 400 so that the conduit member 300 is rotatable with respect to the pad member 400.

The conduit member 300 is coupled with the fluid delivery conduit 131 via the conduit attachment member 309 to: 1) establish fluid communication between a lumen of the fluid delivery line 131 and the lumen 303; and 2) physically attach the conduit member 300 to the fluid delivery conduit 131. As shown, the elbow 307 of the conduit member 300 defines an orientation of the horizontal section 304 to be substantially parallel to a top side 520 of the pad 120. The elbow 307, in turn, defines an orientation of a distal portion 531 of the fluid delivery conduit 131 to parallel to the top side 520. Also shown, the conduit member 300 may be configured to position the distal portion 531 of the fluid delivery conduit 130 immediately adjacent to the top side 520 or in contact with the top side 520. In some embodiments, the conduit member 300 may be configured to position the distal portion 531 of the fluid delivery conduit 130 within about 2 to 10 mm of the top side 520.

As stated above, the vertical section 305 (or at least a portion thereof) of the conduit member 300 may be disposed within the opening 405 of the pad member 400 to facilitate functional interaction between various corresponding features of the conduit member 300 and the pad member 400. As shown, the first outside bearing surface 310 of the conduit member 300 is disposed adjacent to the first inside bearing surface 410 of the pad member 400 to define a face to face relationship between the first outside bearing surface 310 and the first inside bearing surface 410. As such, when the conduit member 300 is rotated with respect to the pad member 400, the first outside bearing surface 310 slidably displaces relative to the first inside bearing surface 410.

Similarly, the second outside bearing surface 312 of the conduit member 300 is disposed adjacent to the second inside bearing surface 412 of the pad member 400 to define a face to face relationship between the second outside bearing surface 312 and the second inside bearing surface 412. As such, when the conduit member 300 is rotated with respect to the pad member 400, the second outside bearing surface 312 slidably displaces relative to the second inside bearing surface 412.

In further similar fashion, the annular sealing surface 320 of the conduit member 300 is positioned adjacent to the groove 420 of the pad member 400 so that a sealing member 525 disposed within the groove 420 defines a fluid seal between the conduit member 300 and the pad member 400. The groove 420 and the sealing member 525 are correspondingly sized to define an interference fit between the sealing member 525 and the annular sealing surface 320. The sealing member 525 in combination with the groove 420 defines a dynamic fluid seal with the annular sealing surface 320 so that when the conduit member 300 is rotated with respect to the pad member 400, the annular sealing surface 320 slidably displaces relative to the sealing member 525. In some embodiments, a lubricant 526 may be disposed between the annular sealing surface 320 and the sealing member 525 to minimize a sliding friction between the annular sealing surface 320 and the sealing member 525.

The sealing member 525 may be formed of a toroidal shape. In some embodiments, the sealing member 525 may define a circular cross-sectional area of revolution such as a torus. In some embodiments, the sealing member 525 may be an O-ring. In other embodiments, sealing member 525 may define a non-circular area of revolution, such as an X-shape. In some embodiments, the sealing member 525 may define a lip seal. Of course, any other shapes of the area of revolution as may be suitable to define the dynamic seal are included in this disclosure. The sealing member may be formed of any suitable sealing material including silicone, rubber, ethylene propylene diene monomer (EPDM), and the like. In some embodiments, the rotatable joint may include more than one sealing member 525.

As will be appreciated by one of ordinary skill, other arrangements of the annular sealing surface 320, the sealing member 525, and the groove 420 may provide for the dynamic fluid seal between the conduit member 300 and the pad member 400 without departing from the spirit of the invention. For example, in some embodiments, the groove 420 may be disposed on the vertical section 305, and the annular sealing surface 320 may be disposed on an inside surface of the opening 405.

In some embodiments, the interaction between the sealing member 525 and the annular sealing surface 320 may define sliding bearing between the conduit member 300 and the pad member 400 similar to the sliding bearing defined first inside bearing surface 410 and the first outside bearing surface 310. In such an embodiment, the sliding bearing defined by the second inside bearing surface 412 and the second outside bearing surface 312 may be omitted.

With further reference to FIG. 5, the deflectable member 330 may be disposed within the recess 430. The hook 332 is shown disposed in overlapping engagement with the ledge 432 to define a snap-fit retention mechanism 530. The retention mechanism 530 prevents separation of the conduit member 300 from the pad member 400 once assembled. Placement of the retention mechanism 530 within the recess 430 may make the deflectable member inaccessible to a user, thereby preventing disassembly by the user.

Assembly of the joint 150 may be performed simply by linearly displacing the conduit member 300 relative to the pad member 400 so that the vertical section 350 of the conduit member 300 is inserted within the opening 405 of the pad member until the hook 332 is inserted beyond the ledge 432. In other words, securing conduit member 300 to the pad member 400 may require only linear displacement of the conduit member 300 relative to the pad member 400. During insertion, the angled surface 333 of the deflectable member 330 may engage with the second inside bearing surface 412 to cause inward deflection of the deflectable member 330, i.e., toward the longitudinal axis 306 or the lumen 303.

With further reference to the FIG. 5, the pad may include multiple layers to provide for multiple functions of the pad 120. A fluid containing layer 550 is shown fluidly coupled with the fluid delivery conduit 131 via the rotatable joint 150 to facilitate circulation of the TTM fluid 112 within the fluid containing layer 550. The pad member 400 is coupled with the fluid containing layer 550 so that the opening 405 is aligned with a fluid inlet 555 of the fluid containing layer 550. The bottom surface 402 of the pad member 400 is scalably attached to a top surface 552 of the fluid containing layer 550. The attachment may include radio-frequency (RF) welding, ultra-sonic welding, adhesive bonding or any other suitable attachment process. The fluid inlet 555 may be coupled with an internal fluid conduit 556 of the fluid containing layer 550 so that TTM fluid 112 entering the fluid containing layer 550 passes through the internal fluid conduit 556.

In some embodiments, the conical structure of the pad member 400 may define a flexibility of the pad member 400 adjacent the circumferential edge 404. In other words, a thickness along the circumferential edge 404 may be sufficiently thin to allow the pad member 400 flex along with the portion of the fluid containing layer 550 attached thereto. The flexibility of the pad member 400 may define a soft connection to the fluid containing layer 550 to form a strain relief between the pad member 400 and fluid containing layer 550 along the circumferential edge 404.

A thermal conduction layer 560 is coupled with the fluid containing layer 550 on a bottom side thereof so as to be disposed between the fluid containing layer 550 and the patient's skin 51 (FIG. 1) during the TTM therapy. An insulation layer 570 is coupled with the top side 552 of the fluid containing layer 550. The insulation layer 570 is configured to inhibit thermal energy transfer between the fluid containing layer 550 and the environment. In some embodiments, the insulation layer 570 may include one or more openings extending through the insulation layer 570 such as the opening 571 to provide for coupling of the fluid conduit 131 with the fluid containing layer 550. More specifically, the rotatable joint 150 may be disposed through the opening 571. In some embodiments, the opening 571 may be conically shaped defining an inside conical surface 573. The inside conical surface 573 may be sized to correspond with the conical surface 403 so that, when the pad 120 is assembled, the inside conical surface 573 is disposed in a face to face relationship with the conical surface 403. In some embodiments, the insulation layer 570 may be attached to the pad member 400.

A method of manufacture of the pad 120 may include one or more of the following steps or process. The conduit member 300 may be attached to the fluid delivery conduit 131 and the pad member 400 may be attached to the fluid containing layer 550. The insulation layer 570 may be attached to the fluid containing layer 550 so that the opening 571 of the insulation layer 570 is aligned with the opening 405 of the pad member 400. The sealing member 525 may be placed within the groove 420. Lubrication 526 may be applied to the sealing member 525 or the annular sealing surface 320 of the conduit member 300. The conduit member 300 may be displaced linearly relative to the pad member 400 so that the vertical section 305 is inserted within the opening 405. During insertion, the deflectable member 330 may deflect inward due to engagement of the angled surface 333 with the second inside bearing surface 412. The insertion may continue until the angled surface 333 disengages the second inside bearing surface 412 allowing the deflectable member 330 to self-deflect outward causing the hook 332 to overlap the ledge 432.

In some embodiments, one or more steps of manufacturing described above may be performed at a first location such as a manufacturing facility, and one or more other steps may be performed at a second location such as a patient care facility. In an embodiment, the steps of attaching the conduit member 300 to the fluid delivery conduit 131, attaching the pad member 400 to the fluid containing layer 550, attaching insulation layer 570 to the fluid containing layer 550, and placing the sealing member 525 within the groove 420 may be performed at the manufacturing facility. The step of linearly displacing the conduit member 300 relative to the pad member 400 so that the vertical section 305 is inserted within the opening 405 may be performed at the patient care facility by the clinician.

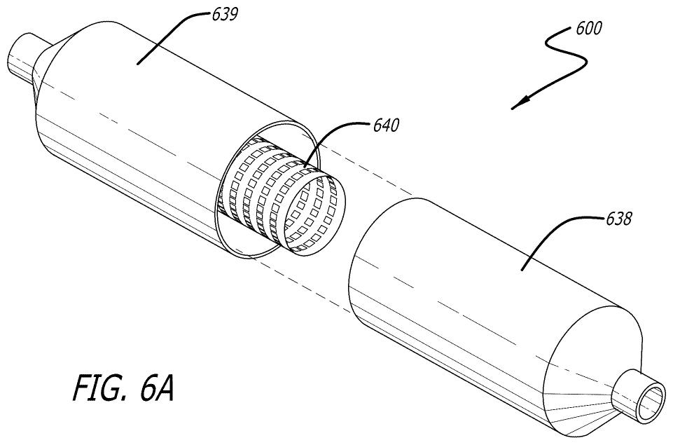

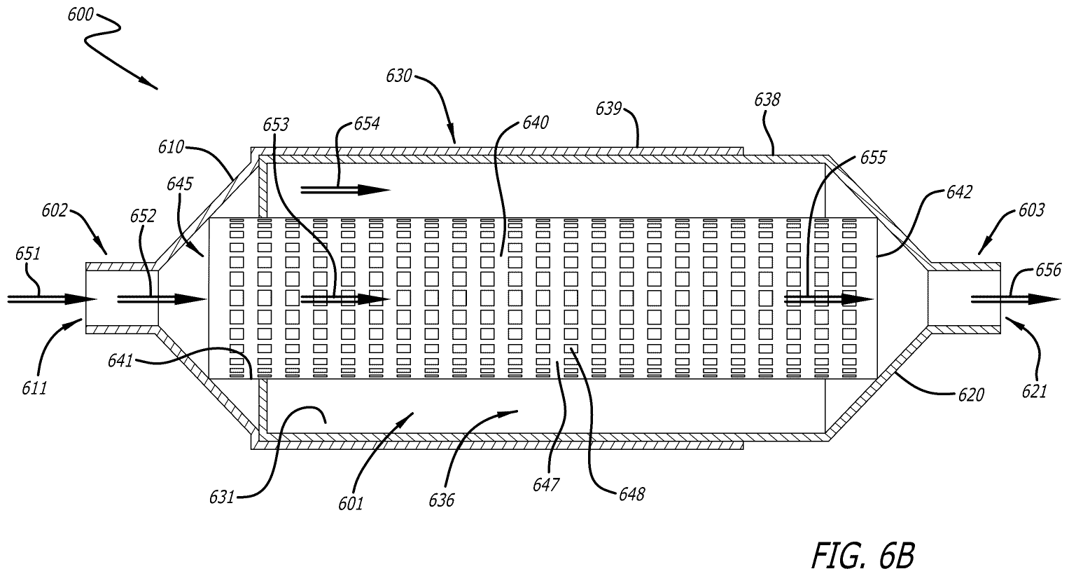

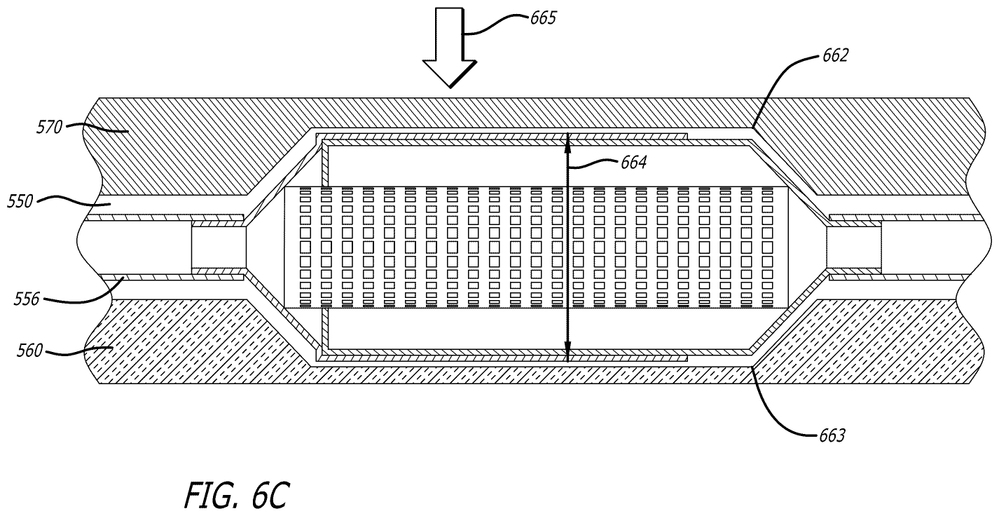

FIGS. 6A and 6B show a filter 600 that may be included with the TTM system 100. The filter 600 may be disposed in line with a TTM fluid flow path of the TTM system 100 so that the circulating TTM fluid 112 flows through the filter 600. The filter 600 may be configured to remove (i.e., filter out) material/particles having a size of 0.2 microns or larger from the TTM fluid 112 without causing a flow restriction of the TTM fluid 112.

The filter 600 includes a longitudinal shape having a flow path 601 extending from a first end 602 to a second end 603. The filter 600 includes a diffuser 610 adjacent the first end 602, a nozzle adjacent 620 the second end 603, and a body 630 extending between the diffuser 610 and the nozzle 620. Along the diffuser 610, a cross-sectional flow area of the filter 600 expands from an inlet flow area 611 to a body flow area 631 and along the nozzle 620, the cross-sectional flow area of the filter 600 contracts from the body flow area 631 to an outlet flow area 621. In some embodiments, the inlet flow area 611 and the outlet flow area 621 may be substantially equal.

In some embodiments, the body flow area 631 may be constant along the body 630. In other embodiments, the body flow area 631 may vary along a length of the body 630 such that the body flow area 631 is greater or less along middle portion of the body 630 than at the ends of the body 630. In some embodiments, the body flow area 631 may be circular.

The filter 600 includes an inner tube 640 disposed within the body 630 extending along the length of body 630. The inner tube 640 may be coupled with the diffuser 610 at a first inner tube end 641 so that TTM fluid 112 entering the filter 600 at the first end 602 also enters the inner tube 640 at the first inner tube end 641. The inner tube 640 may be coupled with the nozzle 620 at a second inner tube end 642 so that TTM fluid 112 exiting the filter 600 at the second end 603 also exits the inner tube 640 at the second inner tube end 642.

The inner tube 640 includes an inner tube flow area 645 extending the length of the inner tube 640. The inner tube flow area 645 may be greater than the inlet flow area 611 and/or the outlet flow area 621. The inner tube flow area 645 may be constant along the length of the inner tube 640. In some embodiments, the inner tube flow area 645 may vary along the length of the inner tube 640. In some embodiments, the inner tube 640 may include a circular cross section. The inner tube 640 and the body 630 may be configured so that the body flow area 631 includes a combination of the inner tube flow area 645 and an annular flow area 636.

The inner tube 640 includes a porous a circumferential wall 647. The porous wall 647 may be configured so that TTM fluid 112 may flow through the porous wall 647, i.e., through the pores 648 of the porous wall 647. Consequently, TTM fluid 112 may flow through the porous wall 647 from the inner tube flow area 645 to the annular flow area 636 and from the annular flow area 636 into the inner tube flow area 645.

In use, the longitudinal velocity of the TTM fluid 112 may change along the length of the filter 600. As the volumetric TTM fluid 112 flow through the filter is constant, the longitudinal velocity of the TTM fluid 112 may be at least partially defined by the flow areas of the filter 600 as described below. The TTM fluid 112 may enter the filter 600 at a first longitudinal velocity 651 and decrease along the diffuser so that the TTM fluid 112 enters the inner tube at a second velocity 652 less than the first longitudinal velocity 651. At this point, a portion of the TTM fluid 112 may flow through the porous wall 647 from the inner tube flow area 645 into the annular flow area 636 to divide the fluid flow into a third velocity 653 within the inner tube flow area 645 and a fourth velocity 654 within the annular flow area 636. The fourth velocity 654 may be less than the third velocity 653. A portion of the TTM fluid 112 may then flow back into the inner tube flow area 645 from the annular flow area 636 to define a fifth velocity 655 along the inner tube flow area 645 which may be about equal to the second velocity 652. The TTM fluid 112 may then proceed along the nozzle 620 to define a sixth velocity 656 exiting the filter 600. In some embodiments, the first velocity 651 and the sixth velocity 656 may be about equal.

The filter 600 may be configured to remove harmful bacteria and viruses from the TTM fluid 112 using sedimentation principles. In use, the filter 600 may be oriented horizontally so that the direction of fluid flow through the filter 600 is perpendicular to a gravitational force 665. In some instances, bacteria, viruses, and other particles within the TTM fluid 112 may have a greater density than the TTM fluid 112 and as such may be urged by the gravitational force 665 (i.e., sink) in a direction perpendicular to the fluid flow direction. In some instances, particles within the inner tube flow area 645 may sink toward and through the porous wall 647 into the annular flow area 636. Particles within the annular flow area 636 may then sink toward an inside surface 631 of the body 630 and become trapped adjacent the inside surface 631. The geometry of the filter 600 may be configured to allow 0.2-micron bacteria/virus particles to fall out of the flow of TTM fluid 112 and become trapped along the inside surface 631.

In some embodiments, the filter 600 may be configured so that flow of TTM fluid 112 from the inner tube flow area 645 into the annual flow area 636 my drag particles through the porous wall 647. In some embodiments, the inlet flow area 611, the inner tube flow area 645, and the annual flow area 636 may be sized so that the third velocity 653 is less than about 50 percent, 25 percent, or 10 percent of the first velocity 651 or less. In some embodiments, the body 630 and the inner tube 640 may be configured so that the fourth velocity 654 is less than the third velocity 653. In some embodiments, the fourth velocity 654 may less than about 50 percent, 25 percent, or 10 percent of the third velocity 653 or less.

In some embodiments, the filter 600 may be configured so that the flow within the inner tube flow area 645 is laminar flow, i.e., so that the velocity of the fluid flow adjacent to or in close proximity to an inside surface 641 of the porous wall 647 is less than the velocity at a location spaced away from the inside surface 641. In such an embodiment, the particles may more readily sink toward and through the porous wall 647.

In some embodiments, the filter 600 may be configured so that the fluid flow within the annual flow area 636 is laminar flow, i.e., so that the velocity of the fluid flow adjacent to or in close proximity to inside surface 631 of the body 630 is less than the velocity at a location spaced away from the inside surface 631. In such an embodiment, the particles may more readily sink toward and be trapped along the inside surface 631.

The filter 600 may include three components including the inner tube 640 an inner body shell 638, and an outer body shell 639. Each of the three components may be formed via the plastic injection molding process. Assembly of the filter 600 may include capturing the inner tube 640 within the inner body shell 638 and the outer body shell 639 and sliding the inner body shell 638 into the outer body shell 639 wherein the fit between the inner body shell 638 and the outer body shell 639 is an interference fit.

In some embodiments, the filter 600 may be disposed within the pad 120. FIG. 6C shows a detail cross-sectional view of the pad 120 including the filter 600 disposed within the fluid containing layer 550. The filter 600 is coupled in line with the internal fluid conduit 556 within the fluid containing layer 550 so that TTM fluid 12 circulating within the pad 120 passes through the filter 600. The filter 600 may be sized so that the inlet flow area 611 and the outlet flow area 621 are similar to a cross-sectional flow area of the internal flow path 660 within the fluid containing layer 550.

In some embodiments, a thickness of the fluid containing layer 550 may increase adjacent the filter 600 to accommodate a body diameter 664 of the filter 600. To further accommodate the body diameter 664, the insulation layer 410 and/or the thermal conduction layer 430 may include internal depressions 662, 663, respectively.

In some embodiments, one or more filters 600 may be disposed in line with the flow of TTM fluid 112 at other locations of the TTM system 100. In some embodiments, one or more filters 600 may be disposed within the TTM module 110. In some embodiments, one or more filters 600 may be disposed in line with the FDL 130. In some embodiments, the filter 600 may be disposed in line with a fluid conduit of the pad external to the fluid containing layer 550 such as a conduit extending between the pad connector 652 and the pad 120.

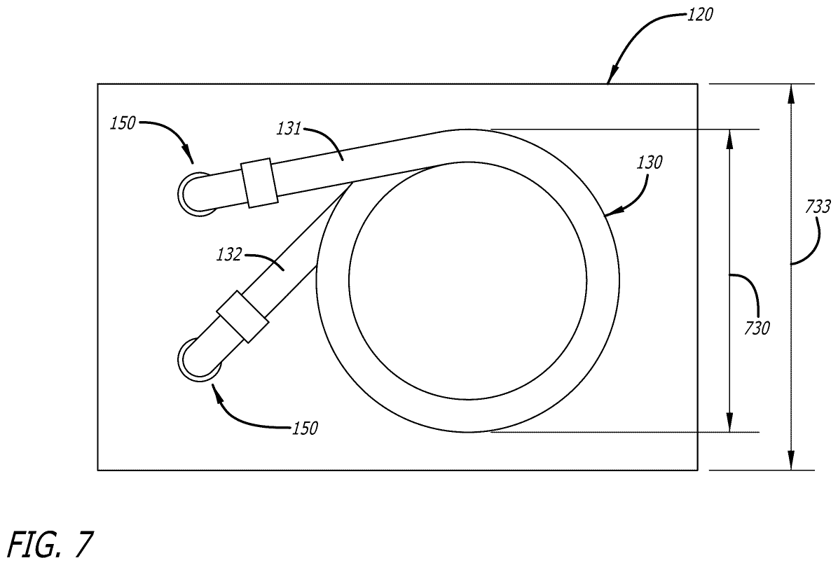

FIG. 7 is a top view of the thermal pad 120 disposed in packaging configuration showing the fluid delivery conduit 131 and the fluid return conduit 132 each coupled with the pad 120 via a rotatable joint 150. The joints 150 have been rotated to orient the fluid delivery conduit 131 and the fluid return conduit 132 in a direction substantially along a side of the pad 120 in contrast to a direction extending away from the pad 120. The fluid deliver line 130 is disposed in a coiled configuration. In some embodiments, the outside diameter 730 of the coil may be less than a width 733 of the pad 120 so that the fluid deliver line 130 is disposed within a circumference of the pad 120. The thermal pad 120 when disposed in the packaging configuration as shown and described, may provide for placement of the pad 120 within a packaging container such as a box or bag having minimal dimensions while maintain the pad 120 in a non-folded state.

A method of using of the pad 120 may include one or more of the following steps or process. The FDL 130 may un-coiled from a coiled packaging state. The FDL 130 including the fluid delivery conduit 131 and the fluid return conduit 132 may be rotated via the rotatable joints 150 to orient the FDL 130 in a first orientation defining a direction of the FDL 130 that is advantageous for coupling the FDL 130 to the TTM module 110 and placement of the pad 120 on the patient 50. The clinician may reposition the pad 120 on the patient 50 away from the first position toward a second position that may include angular rotation of the pad 120. In consequence, the clinician may rotate the FDL 130 away from the first orientation toward a second orientation, where in the second orientation is advantageous for extending the FDL 130 toward the TTM module 110 with the pad 120 disposed in the second position.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. A method of manufacturing a thermal pad for providing thermal energy exchange with a patient, comprising:
including a fluid containing layer with the thermal pad configured for containing a target temperature management (TTM) fluid, wherein the TTM fluid is circulatable within the fluid containing layer from a fluid inlet to a fluid outlet;
coupling a pad joint member to the fluid containing layer pad, the pad joint member having an opening in fluid communication with the fluid inlet of the fluid containing layer; and
inserting a fluid delivery line (FDL) joint member into the opening of the pad joint member in a snap-fit arrangement to engage a retention mechanism between the FDL joint member and the pad joint member, wherein the retention mechanism enables rotation of the FDL joint member with respect to the pad joint member.

2. The method according to claim 1, further comprising coupling a thermal conduction layer to a bottom side of the fluid containing layer.

3. The method according to claim 2, further comprising coupling an insulation layer to top side of the fluid containing layer, wherein the pad joint member is disposed in the insulation layer.

4. The method according to claim 1, further comprising placing a sealing member within a groove of the pad joint member.

5. The method according to claim 4, further comprising applying lubrication to the sealing member.

6. The method according to claim 1, wherein:
the FDL joint member comprises a deflectable member having an angled surface,
the pad joint member includes a contact surface, and
inserting the FDL joint member into the opening of the pad joint member comprises engaging the angled surface with the contact surface to urge the deflectable member away from the contact surface.

7. The method according to claim 1, wherein the pad joint member has a conical outer surface.

8. The method according to claim 1, wherein the FDL joint member comprises a first section disposed orthogonally with respect to a second section, wherein the first section is configured for insertion into the opening of the pad joint member, and wherein the second section is parallel to a top surface of the thermal pad following insertion of the FDL joint member into the pad joint member.

9. The method according to claim 1, wherein the fluid containing layer includes an internal fluid conduit, further comprising disposing a filter inline with the internal fluid conduit, wherein the filter is configured to remove material/particles having a size of 0.2 microns or larger from the TTM fluid.

10. The method according to claim 9, wherein the filter includes a diffuser adjacent a first end and a nozzle adjacent a second end.

11. The method according to claim 10, wherein the filter includes a body between the diffuser and the nozzle, and wherein the body has a cross-sectional flow area larger than the diffuser and the nozzle.

12. The method according to claim 11, wherein the diffuser and the nozzle have a substantially equivalent cross-sectional flow area.

13. The method according to claim 11, wherein the filter includes an inner tube disposed in the body, the inner tube having a cross-sectional flow area less than the cross-sectional flow area of the body and greater than the cross-sectional flow areas of the diffuser and the nozzle.

14. The method according to claim 13, wherein the inner tube includes a porous circumferential wall to permit the TTM fluid to flow through the circumferential wall of the inner tube into an annular flow area surrounding the inner tube.

15. The method according to claim 14, wherein the porous circumferential wall is positioned in the body so that particles with a density greater than a density of the TTM fluid pass into the annular flow area due to gravitational forces.
